(19) United States
(12) Patent Application Publication
(10) Pub. No.: US 2011/0054031 A1
(43) Pub. Date: Mar. 3, 2011

(54) Ophthalmic NSAIDS as Adjuvants

(75) Inventors: Tim McNamara, Irvine, CA (US); Simon P. Chandler, Trabuca Canyon, CA (US); Tetsuo Kida, Osaka (JP)

(73) Assignee: ISTA Pharmaceuticals, Inc., Irvine, CA (US)

(21) Appl. No.: 12/867,470

(22) PCT Filed: Feb. 19, 2009
(86) PCT No.: PCT/US09/34511

§ 371 Date: Nov. 8, 2010

Related U.S. Application Data
(60) Provisional application No. 61/030,464, filed on Feb. 21, 2008.

(51) Int. Cl. A61K 31/196; A61P 27/00; A61P 29/00

(52) U.S. Cl. 514/567

(57) ABSTRACT

The disclosure provides methods and ophthalmic NSAIDs as adjuvants to VEGF inhibitors useful for treating retinal disorders, including but not limited to wet AMD, diabetic retinopathy, diabetic macular edema, central retinal vein occlusion, and branch retinal vein occlusion.

### CROSS-REFERENCES TO RELATED APPLICATIONS

This application claims the benefit of U.S. Provisional Application No. 61/030,464, filed Feb. 21, 2008, which is hereby incorporated by reference in its entirety for all purposes.

### BACKGROUND OF THE DISCLOSURE

Macular degeneration is an incurable eye disease and the leading cause of blindness affecting more than 10 million people aged 55 and older in the United States. This disease is caused by the deterioration of the macula, the central portion of the retina inside the back layer of the eye that focuses, records and sends images from the eye to the brain via the optic nerve. As people age, their chances for developing eye diseases dramatically increases. The specific factors that cause macular degeneration are not conclusively known but research in this area has been increasing.

There are two basic types of macular degeneration: “wet” (exudative) type and “dry” (atrophic) type. Approximately 10-15% of macular degeneration cases are the wet type. This process is also known as wet age-related macular degeneration (wet AMD). In this type, abnormal blood vessels grow under the retina and the macula. This process is commonly known as choroidal neovascularization (CNV). The new blood vessels may bleed and leak fluid, causing the macula to bulge or lift up, thus distorting or destroying central vision. Under these circumstances, vision loss may be rapid and severe. In contrast, the dry type of macular degeneration (dry AMD) does not involve any leakage of blood or serum. Loss of vision may still occur due to deterioration of the retina caused by the formation of small yellow deposits, known as drusen, under the macula. Wet AMD invariably occurs as a function of advanced dry AMD.

Another leading cause of blindness in the United States is due to diabetic retinopathy. Diabetic retinopathy is a common microvascular complication of diabetes, affecting approximately ˜50% of those with diabetes. Of the 209 million Americans over the age of 18 years, diabetic retinopathy affects more than 5.3 million, or a little more than 2.5% of the entire adult US population. Although major advances in the clinical diagnosis and treatment of diabetic retinopathy and its associated complications have been achieved over the past five decades, diabetic retinopathy remains one of the leading causes of new blindness among working-age individuals in developed countries.

Diabetes may lead to a progressive loss of retinal capillaries which results in retinal ischemia. Retinal ischemia is thought to increase the release of growth factors, which subsequently result in abnormal proliferation of new vessels. These vessels are fragile, prone to bleeding, and undergo scarring and fibrosis which can lead to traction on the retina, retinal detachment, and severe visual loss. In addition, many of these growth factors increase retinal vascular permeability, another hallmark of diabetic eye disease. The retinal vessels may also become abnormally permeable at any stage in the disease process. This abnormal permeability results in transudation of blood serum components into the retina and a thickening of the retina called macular edema and affects about half a million people in the United States alone. When this edema involves or threatens the center of the macula, it is called clinically significant macular edema and it can result in visual loss.

The earliest treatment for sealing the leaking vessels in the wet type of macular degeneration, diabetic retinopathy and/or macular edema was with a laser (laser photocoagulation). This was followed by photodynamic therapy (PDT) with Visudyne®, a drug injected intravenously and used to help direct the laser to the affected area. The laser treatment itself however, may cause scarring and the blood vessels may leak again requiring further treatment. Other areas of treatment for these diseases stems from work done in cancer research and the causes of angiogenesis—the growth of new blood vessels. It was discovered that the protein vascular endothelial growth factor (VEGF) is present in the eye and encourages the development of new blood vessels. Drugs have been developed to inhibit VEGF by preventing it from binding with elements that stimulate growth. For example, chemically synthesized short strands of RNA called “aptamers” have been found that bind to VEGF and therefore, prevent the binding of VEGF to its receptor. Currently, there are three VEGF inhibitors in use: Lucentis® (ranibizumab injection), Avastin® (bevacizumab), and Macugen® (pegaptanib sodium injection). All three are given to a patient by intravitreal injection and require that a number of injections be given over an extended period of time. If treatment commences early in the development of the disease, positive results have been shown in slowing progression and in some cases, improving visual acuity. Intravitreal injections however, may involve some degree of risk and/or discomfort to the patient. Some of the side effects of intravitreal injections include the risk of serious eye infection, eye pain, light sensitivity, vision changes, increased eye pressure, retinal detachment, and vitreous floaters. Thus, there remains a need in the art for improved treatments relating to the use of VEGF inhibitors for treating retinal eye disorders such as wet AMD, diabetic retinopathy, diabetic macular edema, central retinal vein occlusion, and branch retinal vein occlusion.

### BRIEF SUMMARY OF THE DISCLOSURE

The disclosure provides methods and ophthalmic NSAIDs as adjuvants to VEGF inhibitors useful for treating retinal disorders, including but not limited to wet AMD, diabetic retinopathy, diabetic macular edema, central retinal vein occlusion, and branch retinal vein occlusion.

Thus, in one aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs.

In another aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the retinal disorder is wet AMD, diabetic retinopathy, diabetic macular edema, central retinal vein occlusion, or branch retinal vein occlusion.

In another aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is bromfenac, diclofenac, flurbiprofen, ketorolac, nepafenac, amfenac, or indomethacin.

In another aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is bromfenac.

In another aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the VEGF inhibitor is bevacizumab, ranibizumab, or pegaptanib.

In another aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is topically administered to the eye.

In another aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is administered before, during or after administration of the VEGF inhibitor.

In another aspect the disclosure provides methods for increasing the interval between intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the interval between intravitreal injections is increased by one or more months. In another aspect, the disclosure provides similar methods wherein the interval between intravitreal injections is increased by two or more weeks. In another aspect, the disclosure provides similar methods wherein the interval between intravitreal injections is increased by one or more weeks.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the retinal disorder is wet AMD, diabetic retinopathy, diabetic macular edema, central retinal vein occlusion, or branch retinal vein occlusion.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is bromfenac, diclofenac, flurbiprofen, ketorolac, nepafenac, amfenac, or indomethacin.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is bromfenac.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the VEGF inhibitor is bevacizumab, ranibizumab, or pegaptanib.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is topically administered to the eye.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the NSAID is administered before, during or after administration of the VEGF inhibitor.

In another aspect the disclosure provides methods for decreasing the number of intravitreal injections in a patient undergoing treatment for a retinal disorder with a VEGF inhibitor to maximize visual acuity, by administering to the patient in need of such treatment, an effective amount of an adjuvant comprising one or more ophthalmic NSAIDs, wherein the number of intravitreal injections is decreased by about half. In other aspects, the disclosure provides similar methods wherein the number of intravitreal injections is decreased from about 30% to about 50%. In other aspects, the disclosure provides similar methods wherein the number of intravitreal injections is decreased from about 10% to about 30%. Still in other aspects, the disclosure provides similar methods wherein the number of intravitreal injections is decreased from about 5% to about 10%.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs, wherein the retinal disorder is wet AMD, diabetic retinopathy, diabetic macular edema, central retinal vein occlusion, or branch retinal vein occlusion.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs, wherein the NSAID is bromfenac, diclofenac, flurbiprofen, ketorolac, nepafenac, amfenac, or indomethacin.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs, wherein the NSAID is bromfenac.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs, wherein the VEGF inhibitor is bevacizumab, ranibizumab, or pegaptanib.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs, wherein the NSAID is topically administered to the eye.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs, wherein the NSAID is administered before, during or after administration of the VEGF inhibitor.

In another aspect the disclosure provides methods for decreasing the amount of a VEGF inhibitor administered by intravitreal injection in a patient undergoing treatment for a retinal disorder with a VEGF inhibitor to maximize visual acuity administering to the patient in need of such treatment, an effective amount of an adjuvant comprising one or more ophthalmic NSAIDs, wherein the amount of the VEGF inhibitor administered by intravitreal injection is decreased by about half. In other aspects, the disclosure provides similar methods wherein the amount of the VEGF inhibitor administered by intravitreal injection is decreased from about 30% to about 50%. In other aspects, the disclosure provides similar methods wherein the amount of the VEGF inhibitor administered by intravitreal injection is decreased from about 10% to about 30%. Still in other aspects, the disclosure provides similar methods wherein the amount of the VEGF inhibitor administered by intravitreal injection is decreased from about 5% to about 10%.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs, wherein the retinal disorder is wet AMD, diabetic retinopathy, diabetic macular edema, central retinal vein occlusion, or branch retinal vein occlusion.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs, wherein the NSAID is bromfenac, diclofenac, flurbiprofen, ketorolac, nepafenac, amfenac, or indomethacin.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs, wherein the NSAID is bromfenac.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs, wherein the VEGF inhibitor is bevacizumab, ranibizumab, or pegaptanib.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs, wherein the NSAID is topically administered to the eye.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs, wherein the NSAID is administered before, during or after administration of the VEGF inhibitor.

In another aspect the disclosure provides methods for decreasing the risk to a patient undergoing intravitreal treatment for a retinal disorder with a VEGF inhibitor to maximize visual acuity, by administering to the patient in need of such treatment, an effective amount of an adjuvant comprising one or more ophthalmic NSAIDs, wherein the risk is infection, pain, light sensitivity, vision changes, increased eye pressure, retinal detachment, vitreous floaters endopthalmitis, or thromboembolic events

### BRIEF DESCRIPTION OF THE DRAWINGS

FIG. 1 illustrates the inhibition of choroidal neovasularization (CNV) lesions after 2 weeks of treatment with topically applied bromfenac ophthalmic solution 0.1% (BF) on mice with CNV induced by laser photo coagulation; and the effect of BF 0.1% with vascular endothelial growth factor (VEGF)-neutralizing protein, recombinant murine soluble receptor 1/Fc chimeric protein (sVEGFR-1/Fc).

### DETAILED DESCRIPTION OF THE DISCLOSURE

### Definitions

“Antimicrobial compound” includes those compounds that effectively kill or mitigate the activity of a microbe. An antimicrobial includes antibacterial, bacteriostatic, and the like. These agents include, but are not limited to: azithromycin, tobramycin, gentamicin, ciprofloxacin, norfloxacin, ofloxacin, and sparfloxacin.

“Derivative” refers to any analog, salt, ester, amine, amide, acid and/or alcohol derived from an active agent of the disclosure which may be used in place of that active agent.

“Diabetic retinopathy” refers to a complication of diabetes typically classified into two stages, Non-Proliferative Diabetic Retinopathy (NPDR) and Proliferative Diabetic Retinopathy (PDR). Diabetic retinopathy is a complication of diabetes that results from damage to the blood vessels of the light-sensitive tissue at the back of the eye (retina). At first, diabetic retinopathy may cause no symptoms or only mild vision problems, eventually however, diabetic retinopathy can result in blindness. In the United States, diabetic retinopathy is a leading cause of blindness in adults. “Non-Proliferative Diabetic Retinopathy” (NPDR) is a complication of diabetes in the early stage of diabetic retinopathy that occurs when normal blood vessels in the retina are damaged due to diabetes and swell and begin to leak fluid and small amounts of blood into the eye.

“Dose” refers to the concentration of the active ingredient (NSAID) or a derivative thereof which may be comprised of an analog, salt, ester, amine, amide, alcohol or acid of the NSAID and may be used in place of the NSAID used. “Lower Dose” formulation comprises the NSAID at a concentration of about 0.05% w/v to about 0.1% w/v, whereas “Higher Dose” formulation comprises the NSAID at a concentration about 0.12% w/v to about 0.24% w/v.

“Eye surface inflammation” includes any inflammatory disorder involving the ocular surface. The eye surface includes the eye lids, conjunctiva and cornea. “Inflammation” refers to white blood cell or leukocytic infiltration associated with cellular injury. Eye surface inflammatory disorders treatable by the ophthalmic preparations of the disclosure are typically manifested by signs and symptoms such as increased cells and flare in the anterior chamber, eye redness, and/or eye irritation. These diseases include, for example, meibomianitis, blepharitis, uveitis, iritis, conjunctival hyperemia, eyelid hyperemia, keratitis and ocular rosacea. The inflammation of tissue associated with the eye may be the result of a number of different causes. Whether the cause is bacterial, viral, traumatic, iatrogenic or environmental, inflammation may be painful, damaging to tissues and requires special care.

“Macular degeneration” refers to a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. It is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

“Macular edema” refers to the swelling of the retina in diabetes mellitus due to leaking of fluid from blood vessels within the macula. The macula is the central portion of the retina, a small area rich in cones, the specialized nerve endings that detect color and upon which daytime vision depends. As macular edema develops, blurring occurs in the middle or just to the side of the central visual field. Visual loss from diabetic macular edema may progress over a period of months and make it impossible to focus clearly.

Macular edema in common in diabetes. The lifetime risk for diabetics to develop macular edema is about 10%. The condition is closely associated with the degree of diabetic retinopathy (retinal disease). Hypertension (high blood pressure) and fluid retention also increase the hydrostatic pressure within capillaries which drives fluid from within the vessels into the retina. A common cause of fluid retention in diabetes is kidney disease with loss of protein in the urine (proteinuria).

Diabetic macular edema is classified into focal and diffuse types. This is an important difference because the two types differ in treatment. Focal macular edema is caused by foci of vascular abnormalities, primarily microaneurysms, which tend to leakage fluid whereas diffuse macular edema is caused by dilated retinal capillaries in the retina.

“Ocular infection” refers to an abnormal condition caused by bacteria, fungi and viruses. Infections, if not treated, can lead to more severe ocular disorders.

“Ocular inflammation” includes, but is not limited to: inflammatory conditions of the palpebral and bulbar conjunctiva, cornea, anterior segment of the globe, and posterior segments of the globe including but not limited to uveitis, scleritis, inflammatory conditions of the retina and macula including but not limited to wet AMD, diabetic retinopathy, diabetic macular edema, central retinal vein occlusion, and branch retinal vein occlusion.

“Ophthalmically-acceptable” refers to the formulation, active agent, excipient or other material is compatible with ocular tissue; that is, it does not cause significant or undue detrimental effects when brought into contact with ocular tissue. In some instances, actives and/or excipients of the formulation may cause some discomfort or stinging in the eye; however, those excipients are still considered ophthalmically-acceptable for the purposes of this application. In many instances, these irritating components are removed from the formulations for comfort of the patient. For example, polyvinyl alcohol (PVA) may be eliminated from the formulation ingredients.

A “patient” refers to a vertebrate, typically a mammal, more typically a human. Mammals include, but are not limited to: humans, rodents, sport animals and pets, such as rats, dogs, and horses.

“Therapeutically-active agent” refers to any agent capable of having a therapeutic effect.

“Therapeutically-effective amount” refers to an amount of active sufficient to prevent, inhibit, or reduce the level of inflammation, irritation or other abnormal conditions in the eye.

### General

Macular degeneration may be caused by a variety of conditions, including but not limited to myopia, presumed ocular histoplasmosis syndrome (POHS), genetic predisposition, and aging. Blindness in macular degeneration is most often the result of the growth of abnormal blood vessels beneath the retina called the choroidal neovascular membrane. These membranes leak, hence the name “wet” macula degeneration. There are several types of symptomatic treatment, however, that have been used with limited and isolated success, depending on the particular condition of the patient, to treat exudative (wet form) macular degeneration. Laser photocoagulation therapy may benefit certain patients with macular degeneration. However, there are high recurrence rates for selected choroidal neovascular membranes which may initially respond to laser therapy. Vision loss may also result from the laser therapy. Low dose radiation (teletherapy) has also been hypothesized as a possible treatment to induce regression of choroidal neovascularization. Surgical removal of neovascular membranes is another possible treatment, but it is a highly specialized procedure and reportedly has not had promising results to date. There is presently no effective treatment for non-exudative (dry form) macular degeneration. Management of non-exudative macular degeneration is limited to early diagnosis and careful follow-up to determine if the patient develops choroidal neovascularization. Protection against exposure to ultraviolet light and prescribed dosages of anti-oxidant vitamins (e.g., vitamin A, β-carotene, lutein, zeaxanthin, vitamin C and vitamin E) and zinc may also be of some benefit, but as yet these treatments remain unproven. Accordingly, the population to be treated by the disclosed methods includes (i) a human subject diagnosed as suffering from macular degeneration, (ii) a human subject diagnosed as suffering from diabetes-related retinopathy, and (iii) a human subject suffering from pathological vascularization of the cornea secondary to injury or disease.

Retinal vein occlusion refers to the closure of the central retinal vein that drains the retina or to that of one of its branches. Retinal vein occlusion (RVO) occurs when the central retinal vein, the blood vessel that drains the retina, or one of its branches becomes blocked. RVO may be categorized by the anatomy of the occluded vein and the degree of ischemia produced. The two major RVO types are central retinal vein occlusion (CRVO) and branch retinal vein occlusion (BRVO). CRVO has been diagnosed in patients as young as nine months to patients of 90 years. The age of affected individuals is usually low to mid 60s. Approximately 90% of patients are over 50 at the time of diagnosis, with 57% of them being male and 43% being female. BRVO accounts for some 30% of all vein occlusions.

CRVO is a painless loss of vision that can be caused by a swollen optic disk, the small area in the retina where the optic nerve enters the eye, by dilated retinal veins, and by retinal hemorrhages. CRVO is also called venous stasis retinopathy, or hemorrhagic retinopathy. In BRVO, the superotemporal branch vein is the most often affected vessel. Retinal hemorrhages follows, often occurring at the crossing of two vessels near the optic disk. Initially the hemorrhage may be extensive and underlie the fovea.

The exact cause of RVO is not yet identified, but the following mechanisms been proposed including but not limited to: external compression between the central connective strand and the cribriform plate; venous disease; and blood clot formation. Conditions associated with RVO risk include: hypertension; hyperlipidemia; diabetes mellitus; hyperviscosity; hypercoagulability; glaucoma; and trauma.

A complete physical evaluation is recommended for CRVO and BRVO, including but not limited to complete blood tests, and glucose tolerance test (for non-diabetics). In the case of a head injury when bleeding around the optic nerve is a possibility, an MRI may be performed. Following a patient with RVO is vital. Patients should be seen at least monthly for the first three months to monitor for signs of other complications, such as the abnormal formation of blood vessels (neovascularization) in the iris of the eye or glaucoma.

The treatment for retinal vein occlusion varies for each case and should be given based on the doctor's best recommendation. Although treatments for occlusion itself are limited, surgical treatment of the occlusion provides an option. Treatments may include anticoagulants with heparin, bishydroxycoumarin, and streptokinase. When the blood is highly viscous, dilution of the blood may be useful. Ideally, an alternate pathway is needed to allow venous drainage. Recent reports published in 1999 suggest that use of a laser to create a retinal choroidal hole may be useful to treat CRVO. Laser therapy depends on the type of occlusion. The management of laser therapy should be controlled by an ophthalmologist. The outlook for people with RVO is fairly good whether it is treated early or not. With no treatment at all, approximately 60% of all patients recover 20/40 vision or better within a year.

Vascular endothelial growth factor (VEGF) has been found to be a key player in the development of choroidal neovascularization (CNV) associated with retinal disorders such as wet macular degeneration. As a result, anti-VEGF therapy is now the cornerstone treatment for wet age-related macular degeneration (wet AMD) and includes the off-label use of the full sized anti-VEGF antibody bevacizumab (Avastin® (bevacizumab), Genentech, Inc.), the on-label use of the genetically engineered antibody fragment ranibizumab (Lucentis® (ranibizumab injection), Genentech, Inc.), and the oligonucleotide “aptamer” pegaptanib sodium (Macugen® (pegaptanib sodium injection), OSI/Eyetech).

NSAIDs are inhibitors of the cyclooxygenase enzymes, COX-1 and COX-2, which synthesize prostaglandins and act to inhibit angiogenesis essential for tissue repair and cancer growth. The molecular mechanism of NSAIDs inhibition of angiogenesis remain unexplained. However, NSAID inhibition of hypoxia-induced angiogenesis may play a role in treatment of wet AMD. In particular, NSAIDs may affect expression levels of the hypoxia-inducible transcription factor-1α (HIF-1α) and/or the von Hippel Lindau tumor suppressor (VHL). In turn, HIF-1α and VHL control hypoxia-induced expression of VEGF (the most potent angiogenic factor) and its specific receptor, Flt-1, both major mediators of hypoxia-induced angiogenesis. Under hypoxia, VHL expression levels are suppressed leading to HIF-α accumulation, VEGF/Flt-1 expression, and angiogenesis. In the presence of NSAIDs, VHL is up-regulated leading to increased ubiquitination and degradation of HIF-1α, causing reduced VEGF/Flt-1 expression and inhibition of hypoxia-induced angiogenesis. The soluble VEGF receptor by definition, binds the secreted or extracellular-membrane bound forms of VEGF-A protein. The potential therefore exists, that these two agents (i.e., NSAIDS and VEGF inhibitors), may work in combination, one reducing mRNA pools of VEGF-A and the other depleting available pools of VEGF-A protein.

Current therapies for ocular angiogenic disease center on the administration of agents by intravitreal injection. The risks associated with this process are manifold and serious, including but not limited to endopthalmitis, retinal detachment and thromboembolic events in older at-risk patients. The above combinations would allow for a reduction in the number of intravitreal injections while preserving or enhancing the efficacy of the therapy. This may increase patient acceptance of this invasive therapy, reduce the impact on ophthalmic clinics with fewer injections per patient and may enhance patient-specific response compared to intravitreal VEGF treatment alone.

### VEGF Inhibitors

Lucentis® (ranibizumab injection) is a prescription medicine available from Genentech, Inc., for the treatment of patients with wet age-related macular degeneration (wet AMD). Ranibizumab has a molecular weight of approximately 48 kilodaltons and is produced by an E. coli expression system in a nutrient medium containing the antibiotic tetracycline. The active ingredient, ranibizumab, is a recombinant humanized IgG1 kappa isotype monoclonal antibody fragment designed for intravitreal use.

Vascular endothelial growth factor A (VEGF-A) has been shown to cause neovascularization in models of ocular angiogenesis and is thought to contribute to the progression of the neovascular form of wet AMD. Ranibizumab acts by binding to and inhibiting the biologic activity of VEGF-A. In particular, ranibizumab binds to the receptor binding site of active forms of VEGF-A, including but not limited to the biologically active, cleaved form of this molecule, VEGF110. The binding of ranibizumab to VEGF-A prevents the interaction of VEGF-A with its receptors (VEGFR1 and VEGFR2) on the surface of endothelial cells, thus reducing endothelial cell proliferation, vascular leakage, and new blood vessel formation.

Lucentis® (ranibizumab injection) is available as a single-use glass vial designed for monthly intravitreal injections of 0.05 mL of 10 mg/mL solution (0.5 mg ranibizumab). Although less effective, treatment may be reduced to one injection every three months after the first four injections if monthly injections are not feasible. Each vial should only be used for the treatment of a single eye. If the contralateral eye requires treatment, a new vial should be used and the sterile field, syringe, gloves, drapes, eyelid speculum, filter, and injection needles should be changed before Lucentis® (ranibizumab injection) is administered to the other eye.

The intravitreal injection procedure should be carried out under controlled aseptic conditions, including but not limited to the use of sterile gloves, a sterile drape, and a sterile eyelid speculum (or equivalent). Adequate anesthesia and a broad spectrum microbicide should be given prior to the injection. Each Lucentis® (ranibizumab injection) carton (NDC 50242-080-01) contains a 0.2 mL fill of 10 mg/mL ranibizumab in a 2-cc glass vial; one 5-micron, 19-gauge×1½ inch filter needle for withdrawal of the vial contents; one 30-gauge×½ inch injection needle for the intravitreal injection; and one package insert. Using aseptic techniques, all (0.2 mL) of the Lucentis® (ranibizumab injection) vial contents are withdrawn through a 5-micron, 19-gauge filter needle attached to a 1-cc tuberculin syringe. The filter needle should be discarded after withdrawal of the vial contents and should not be used for intravitreal injection. The filter needle should be replaced with a sterile 30-gauge×½-inch needle for the intravitreal injection. The contents should be expelled until the plunger tip is aligned with the line that marks 0.05 mL on the syringe.

Following the intravitreal injection, patients should be monitored for elevation in intraocular pressure and for endophthalmitis. Monitoring may consist of a check for perfusion of the optic nerve head immediately after the injection, tonometry within 30 minutes following the injection, and biomicroscopy between two and seven days following the injection. Patients should be instructed to report any symptoms suggestive of endophthalmitis without delay. In addition, patients should be monitored during the week following the injection to permit early treatment should an infection occur. Lucentis® (ranibizumab injection) is contraindicated in patients with ocular or periocular infections as well as in patients with known hypersensitivity to ranibizumab or any of the excipients in Lucentis® (ranibizumab injection). Hyper-sensitivity reactions may be manifested as severe intraocular inflammation. Increases in intraocular pressure have been noted within 60 minutes of intravitreal injection with Lucentis® (ranibizumab injection). Although a low rate (<4%) of arterial thromboembolic events was observed in the Lucentis® (ranibizumab injection) clinical trials, there is a theoretical risk of arterial thromboembolic events following intraocular use of inhibitors of VEGF.

Serious adverse reactions related to the intravitreal injection procedure for Lucentis® (ranibizumab injection) occur in <0.1% of injections. These reactions included endophthalmitis, rhegmatogenous retinal detachments, and iatrogenic traumatic cataracts. Other serious ocular adverse reactions observed among Lucentis® (ranibizumab injection) treated patients occurring in <2% of patients. These reactions included intraocular inflammation and increased intraocular pressure. The most frequently reported ocular adverse reactions that were reported with Lucentis® (ranibizumab injection) treatment include are conjunctival hemorrhage, eye pain, vitreous floaters, increased intravitreal pressure, intraocular inflammation, eye irritation, cataract, foreign body sensation in eyes, increased lacrimation, eye pruritis, visual disturbance, blepharitis, ocular hyperemia, maculopathy, dry eye, ocular discomfort, conjunctival hyperemia, and posterior capsule opacification.

Avastin® (bevacizumab) is also a prescription medicine available from Genentech, Inc. While this medication only has FDA approval for treating colon and rectal cancers, many eye doctors have been using Avastin® (bevacizumab) as an off-label treatment for wet AMD (Lucentis® (ranibizumab injection) is a shorter peptide than Avastin® (bevacizumab) and this difference is thought to give Lucentis® (ranibizumab injection) an advantage in its ability to penetrate the eye's retina and halt abnormal blood vessel growth contributing to advanced macular degeneration and scarring that causes blindness). Bevacizumab, the active ingredient in Avastin® (bevacizumab), is a recombinant humanized monoclonal IgG1 antibody, which has a molecular weight of approximately 149 kilodaltons, and is produced in a Chinese Hamster Ovary mammalian cell expression system in a nutrient medium containing the antibiotic gentamicin. Bevacizumab also contains both human framework regions and the complementarity-determining regions of a murine antibody that binds to VEGF. The interaction of VEGF with its receptors is believed to lead to endothelial cell proliferation and new blood vessel formation in in-vitro models of angiogenesis. It has been found that bevacizumab binds to and inhibits the biologic activity of human VEGF in in-vitro and in-vivo assay systems. In particular, bevacizumab binds VEGF and prevents the interaction of VEGF to its receptors (Flt-1 and KDR) on the surface of endothelial cells.

Avastin® (bevacizumab) is a clear to slightly opalescent, colorless to pale brown, sterile, pH 6.2 solution for intravenous (IV) infusion. It is supplied in 100 mg and 400 mg preservative free, single use vials to deliver 4 mL or 16 mL of Avastin® (bevacizumab) (25 mg/mL). The 100 mg product is formulated in 240 mg α,α-trehalose dihydrate, 23.2 mg sodium phosphate (monobasic, monohydrate), 4.8 mg sodium phosphate (dibasic, anhydrous), 1.6 mg polysorbate 20, and water for injection, USP. The 400 mg product is formulated in 960 mg α,α-trehalose dihydrate, 92.8 mg sodium phosphate (monobasic, monohydrate), 19.2 mg sodium phosphate (dibasic, anhydrous), 6.4 mg polysorbate 20, and water for injection, USP.

Macugen® (pegaptanib sodium injection) is indicated in the United States for the treatment of wet AMD and is administered by intravitreal injection once every six weeks in a 0.3 mg dose. VEGF is a protein that plays a critical role in angiogenesis (the formation of new blood vessels) and increased permeability (leakage from blood vessels), two of the pathological processes that contribute to the vision loss associated with wet AMD. Macugen® (pegaptanib sodium injection) is a pegylated anti-VEGF aptamer, which binds to and inhibits the interaction of VEGF with its receptor.

Macugen® (pegaptanib sodium injection) is contraindicated in patients with ocular or periocular infections. Intravitreal injections including but not limited to those with Macugen® (pegaptanib sodium injection) have been associated with endophthalmitis. Proper aseptic injection technique—which includes use of sterile gloves, a sterile drape, and a sterile eyelid speculum (or equivalent)—should always be utilized when administering Macugen® (pegaptanib sodium injection). In addition, patients should be monitored during the week following the injection to permit early treatment, should an infection occur.

Increases in intraocular pressure (IOP) have been seen within 30 minutes of injection with Macugen® (pegaptanib sodium injection). Therefore, IOP as well as the perfusion of the optic nerve head should be monitored and managed appropriately. Serious adverse events related to the injection procedure occurring in <1% of intravitreal injections included endophthalmitis, retinal detachment, and iatrogenic traumatic cataract. Most frequently reported adverse events in patients treated for up to 2 years were anterior chamber inflammation, blurred vision, cataract, conjunctival hemorrhage, conical edema, eye discharge, eye irritation, eye pain, hypertension, increased IOP, ocular discomfort, punctate keratitis, reduced visual acuity, visual disturbance, vitreous floaters, and vitreous opacities. These events occurred in approximately 10% to 40% of patients.

Associated with all three VEGF inhibitors, i.e., Avastin® (bevacizumab), Lucentis® (ranibizumab injection) and Macugen® (pegaptanib sodium injection), may be the increased risk of stroke and/or cardiovascular events.

### NSAIDs

NSAIDs have been shown to play a role in treating various eye conditions and diseases. Several NSAIDs have been approved for the treatment of a variety of anterior segment conditions including but not limited to post-operative inflammation following cataract surgery, prevention of miosis during cataract surgery, and post-operative pain following refractive and cataract surgery. NSAIDs may also be effective as primary or adjunctive therapy for the treatment of posterior segment disorders. For example, NSAIDs have been shown to be effective both alone and as adjunctive therapy with steroids for treating pseudophakic cystoid macular edema (CME). NSAIDs have also been used off-label as an adjunctive therapy for macular edema associated with diabetic retinopathy, retinal vein occlusions, uveitis, choroidal neovas ularization and epiretinal membranes.

All NSAIDs produce anti-inflammatory and analgesic effects by inhibiting the activity of cyclooxygenases (COX enzymes), enzymes that convert arachidonic acid to cyclic endoperoxides, thereby blocking synthesis of prostaglandins. Prostaglandins mediate many forms of systemic and localized inflammation including but not limited to inflammation in ocular tissues. In animal models, prostaglandins have been shown to produce disruption of the blood-aqueous humor barrier, vasodilation, increased vascular permeability, and leukocytosis.

There are two important isoforms of the COX enzyme: COX-1 and COX-2. COX-1 is an enzyme that is expressed constitutively in almost all tissues, particularly in the gastro-intestinal tract, platelets, endothelial cells, and kidneys. COX-1 catalyzes the production of arachidonic acid into cytoprotective prostaglandins that coat the stomach lining with mucus (gastrointestinal or GI protection) and mediate platelet aggregation. The expression of COX-2 occurs in response to the exposure to a noxious stimulus and leads to the production of prostaglandins that cause inflammation and pain. COX-2 catalyzes the conversion of arachidonic acid into the inflammatory prostaglandins involved in postoperative inflammation, uveitis, allergic conjunctivitis, pupillary miosis, and cystoid macular edema (CME). It has been demonstrated in rats that COX-2 is the primary mediator for ocular inflammation and is thought to be the most important therapeutic mechanism of ophthalmic NSAIDs.

The major factors affecting selection of ophthalmic NSAIDs include safety, efficacy, dosing frequency, tolerance, costs and availability. The safety of these ophthalmic NSAIDs appears to be excellent, although there have been reports of significant side effects such as corneal epitheliopathy, corneal mels, allergic conjunctivitis and systemic effects such as GI upset and prolonged bleeding times. There is also evidence that ophthalmic NSAIDs may interfere with the intraocular pressure-lowering effects of prostaglandins such as latanoprost.

Ophthalmic NSAIDs have become the cornerstone for managing ocular pain and inflammation. Their well characterized anti-inflammatory activity, analgesic property, and established safety record have also made ophthalmic NSAIDs an important tool for optimizing surgical outcomes. Ophthalmic NSAIDs currently play four principal roles in ophthalmic surgery, including but not limited to the prevention of intra-operative miosis during cataract surgery, management of postoperative inflammation, the reduction of pain and discomfort after cataract and refractive surgery, and the prevention and treatment of cystoid macular edema (CME) after cataract surgery. In clinical practice, ophthalmic NSAID therapy provides a highly effective anti-inflammatory activity, a rapid onset of action that produces sustained relief of inflammation and pain, an excellent safety profile, a formulation that is comfortable and well tolerated, and a convenient dose regimen. Commonly used ophthalmic NSAIDs include Acular® (ketorolac tromethanine 0.5%, Allergan, Inc.); Xibrom® (bromfenac 0.09%, Ista Pharmaceuticals, Inc.); Nevanac® (nepanac 0.1% suspension, Alcon, Inc.); Ocufen® (flurbiprofen sodium 0.03%, Novartis AG).

The approved indications and the relative potency of several ocular NSAIDs are provided in Table 1:

TABLE 1
Indications and Relative Potency of Ocular NSAIDs

| | | | | IC₅₀ μM | | |
|---|---|---|---|---|---|---|
| | Brand | Approved | | COX-1 & | | Ratio |
| Drug | Name | Indication(s) | Dosing | COX-2 | | COX-1/COX-2 |
| bromfenac | Xibrom ® | pain and | b.i.d. | 0.53 | 0.023 | 23.0 |
| | | inflammation | | | | |
| diclofenac | Voltaren ® | pain and | q.i.d. | 0.95 | 0.085 | 11.2 |
| | | inflammation | | | | |
| flurbiprofen | Ocufen ® | miosis | q.i.d. | 0.082 | 0.102 | 0.0803 |
| ketorolac | Acular ®, | pain and | q.i.d. | 0.02 | 0.12 | 0.167 |
| | Acular ® LS | inflammation | | | | |
| nepafenac* | Nevanac ® | inflammation | t.i.d. | 0.25 | 0.15 | 1.67 |
| indomethacin | Indocin ®** | inflammation | q.i.d. | 0.28 | 1.68 | 0.167 |

As shown in Table 1, ocular drugs bromfenac, diclofenac, flurbiprofen, ketorolac, nepafenac and indomethacin all have activity against the COX-1 and COX-2 enzymes. The lower the IC₅₀ values, the higher the potency of the ocular drug. Moreover, the higher the ratio of COX-1/COX-2, the higher the theoretical effect on inflammation versus platelet inhibition and gastrointestinal prophylaxis. (*The active form of nepafenac is amfenac and the IC₅₀, shown are for amfenac. **Indoein® is not commercially available in the United States but may be formulated as a 0.1% solution by a compounding pharmacy.)

NSAIDs vary in their relative potency against COX-1 and COX-2. Relative potency is assessed by determining the concentration of drug required to inhibit the COX enzyme activity by 50%, a value called the inhibitory concentration 50% or IC50. A smaller IC50 value signifies greater inhibition of the enzyme (i.e., a lower concentration of drug is needed to inhibit the enzyme). Several in vitro assays are used to determine IC50 values, making the values dependent upon the animal model (tissue and stimulus) used in the experiment and variable between laboratories. Thus, it is important that the assay type is defined when making comparisons between IC50 measurements.

Acular® (ketorolac tromethamine ophthalmic solution) is a member of the pyrrolo-pyrrole group of nonsteroidal anti-inflammatory drugs (NSAIDs) for ophthalmic use. Its chemical name is (±)-5-benzoyl-2,3-dihydro-1H pyrrolizine-1-carboxylic acid, 2-amino-2-(hydroxymethyl)-1,3-propanediol salt (1:1) and has the following structure:

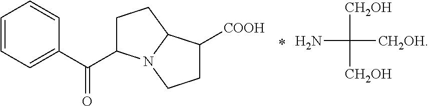

Acular® ophthalmic solution is supplied as a sterile isotonic aqueous 0.5% solution, with a pH of 7.4. Acular® ophthalmic solution is a racemic mixture of R-(+) and S-(−)-ketorolac tromethamine. Ketorolac tromethamine may exist in three crystal forms. All forms are equally soluble in water. The pKa of ketorolac is 3.5. This white to off-white crystalline substance discolors on prolonged exposure to light. The molecular weight of ketorolac tromethamine is 376.41. The osmolality of Acular® ophthalmic solution is 290 mOsml/kg. Each mL of Acular® ophthalmic solution contains as the active ingredient: ketorolac tromethamine 0.5%; and the inactive ingredients: benzalkonium chloride 0.01% (preservative); edetate disodium 0.1%; octoxynol 40; purified water; sodium chloride; and hydrochloric acid and/or sodium hydroxide to adjust the pH.

Xibrom® (bromfenac ophthalmic solution) 0.09% is a sterile, topical, nonsteroidal anti-inflammatory drug (NSAID) for ophthalmic use. Each mL of Xibrom® contains 1.035 mg bromfenac sodium equivalent to 0.9 mg bromfenac free acid. Bromfenac sodium is designated chemically as sodium 2-amino-3-(4-bromobenzoyl)phenylacetate sesquihydrate, with an empirical formula of C₁₅H₁₁BrNNaO₃.1½H₂O, and has the structural formula:

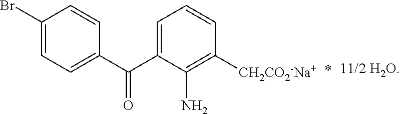

Bromfenac is described in U.S. Pat. Nos. 4,910,225, 5,603,929, 5,653,972, and in U.S. Patent Application Publication Nos. 20050239895 and 20070287749, the disclosures of each of which are hereby incorporated by reference in their entirety for all purposes. Bromfenac sodium salt is a yellow to orange crystalline powder. The molecular weight of bromfenac sodium is 383.17 g/mole. Xibrom® ophthalmic solution is supplied as a sterile aqueous 0.09% solution, with a pH of 8.3. The osmolality of Xibrom® ophthalmic solution is approximately 300 mOsmol/kg. Each mL of Xibrom® ophthalmic solution contains as the active ingredient: bromfenac sodium hydrate 0.1035%; and the inactive ingredients: benzalkonium chloride (0.05 mg/mL) (preservative), boric acid, disodium edetate (0.2 mg/mL), polysorbate 80 (1.5 mg/mL), povidone (20 mg/mL), sodium borate, sodium sulfite anhydrous (2 mg/mL), sodium hydroxide to adjust the pH, and purified water, USP.

The chemical structure of bromfenac is similar to amfenac, the active form of the prodrug nepafenac, except for the key addition of a bromine atom in the 4-position of the benzoyl ring. Importantly, compounds that contain a halogen have greater potency (I˜Br>Cl>F>H). The addition of bromine to the bromfenac molecule imparts more pronounced effects on its in vitro and in vivo potency, absorption across the cornea, and penetration into ocular tissues. Preclinical data confirm that the unique bromine moiety in bromfenac enhances both the in vitro potency of the molecule and the tissue penetration of the ophthalmic formulation.

Bromfenac sodium ophthalmic solution 0.1% was first approved in May 2000 as Bronuck (Senju Pharmaceutical Company, Ltd., Osaka, Japan) and is presently approved by the Ministry of Health in Japan for the clinical indications of the treatment of postoperative inflammation, blepharitis, conjunctivitis, and scleritis. The same formulation was approved in the United States by the Food and Drug Administration (FDA) in March 2005 as Xibrom® (bromfenac ophthalmic solution 0.09%) for the treatment of postoperative inflammation in patients who have undergone cataract extraction. Despite the stated difference in concentrations, the strength of Bronuck 0.1% is equivalent to Xibrom® 0.09%. In January 2006, the FDA-approved indication for Xibrom® was expanded to include the reduction of ocular pain after cataract extraction. Xibrom® is the first and only ophthalmic NSAID with an approved twice-daily dosage.

As a result of its chemical structure, bromfenac has been shown to be the most potent ophthalmic NSAID in inhibiting the COX-2 enzyme. In vitro studies have shown that the inhibition of prostaglandin synthesis with bromfenac was approximately 12 times greater than that of indomethacin. The inhibitory effects of bromfenac on COX-2 have been shown to be 3.7 times greater than diclofenac, 6.5 times greater than amfenac, and 18 times more potent than ketorolac. The COX-2 purified from rabbit alveolar macrophage was used for the COX-2 enzyme inhibition assay of bromfenac, diclofenac, andamfenac. COX activity of ketorolac and bromfenac was determined by measuring prostaglandin-2 production after incubating with human recombinants COX-2 and arachidonic acid. The ability to penetrate ocular tissues may be an important determinant of the efficacy of an ophthalmic NSAID. Studies with bromfenac ophthalmic solution in both animals and humans have demonstrated that the drug penetrates rapidly and extensively into all ocular tissues after ophthalmic application.

Bromfenac was originally developed as a topically applicable drug with lesser side effects and with superior effectiveness in the treatment of ocular inflammation than steroidal anti-inflammatory agents. Bromfenac is a benzoylphenylacetic acid derivative that is very effective in the treatment of inflammatory ophthalmopathy, especially of uveitis, by topical application, and that the effectiveness is compatible with that of conventional steroid anti-inflammatory drugs. Furthermore, bromfenac has been found to be stable in aqueous solutions with the optimal pH range for a locally administrable therapeutic composition.

When topically administered to the eye, a medicinal agent such as bromfenac has to pass through the cornea so that it can reach the site of inflammation or in the case of wet AMD, the retina. After arriving at these sites, bromfenac has been found to remain there in a necessary concentration for a necessary period of time to be effective while not being irritating to the eye. Furthermore, in case of administration in the form of eye drops, bromfenac solutions have been found to be stable for a long period of time in an aqueous solution without decomposition or forming insoluble matters.

Bromfenac has been found to be stable in the salt form. These salts includes alkali metal salts such as sodium salt and potassium salt, alkaline earth metal salts such as calcium salt and magnesium salt, among others, and any salt may suitably be used provided that it can attain the object of the disclosure. Bromfenac may also be obtained in the form of a hydrate depending on the conditions of synthesis, recrystallization and so forth. Bromfenac has also been found to be stable in aqueous solutions by incorporating a water-soluble polymer and sulfite and adjusting the pH to about 6-9.

Bromfenac as the active ingredient in the topically administrable therapeutic compositions for inflammatory eye disease as well as an adjuvant for wet AMD may be produced as described in the Journal of Medicinal Chemistry, 27, p 1379-1388 (1984) or U.S. Pat. No. 4,045,576, or by a modification of the method described therein. Bromfenac may be formulated in an ophthalmic composition which may be prepared in the form of eye drops, eye ointments and so on for topical administration to the eye. Thus, bromfenac may be formulated in an aqueous or non-aqueous solution or mixed with an ointment base suited for ophthalmic use. An aqueous base generally used in the production of ophthalmic preparations, for example sterile distilled water, is suitably used as the aqueous base and the pH thereof is adjusted to a level suited for topical administration to the eye. An appropriate buffer may be added in adjusting the pH. The pH of the ophthalmic compositions may be selected with due consideration paid to the stability and topical eye irritativity of the active ingredient. The stability of an aqueous composition containing bromfenac may be enhanced by incorporating a water-soluble polymer and sulfite, and adjusting the pH to 6.0-9.0, typically with the pH range of 7.5-8.5. The eye irritation of the solution is not observed. Useful water-soluble polymer includes polyvinyl pyrrolidone, carboxypropylcellulose, hydroxyethylcellulose, hydroxypropylcellulose, polyvinyl alcohol, sodium salt of polyacrylic acid an so on. The concentration of the water-soluble polymer may be in the range of about 0.1 to 10 w/w %. Sulfite may include sodium, potassium, magnesium, calcium salt and so on. The concentration of sulfite may be in the range of about 0.1 to 1.0 w/w %. The pH adjustment is generally conducted with sodium hydroxide or hydrochloric acid, for instance, and it is advisable to form a buffer solution by combined use of, for example, sodium acetate, sodium borate or sodium phosphate and acetic acid, boric acid or phosphoric acid, respectively. The ophthalmic compositions may further contain pharmaceutically active ingredients, such as an anti-inflammatory agent of another kind, an analgesic and an antimicrobial compound. Examples of antiinflammatory agents include indomethacin and pranoprofen. Usable examples of the antimicrobial agents are penicillins, cephalosporins, and synthesized antimicrobial agents of the quinolonecarboxylic acid series. Among these, bromfenac may be synergistic with any of these additional ingredients. The analgesic is suited for the purpose of alleviating inflammation-associated pain, and the antimicrobial agent is suited for the purpose of preventing secondary infection. It is of course possible to incorporate active agents other than those mentioned above in the ophthalmic compositions.

In preparing the ophthalmic compositions, an isotonizing agent, a microbicidal agent or preservative, a chelating agent, a thickening agent and so forth may be added to the compositions in accordance with the general practice of ophthalmic preparation manufacture. The isotonizing agent includes, among others, sorbitol, glycerine, polyethylene glycol, propylene glycol, glucose and sodium chloride. The preservative includes, among others, para-oxybenzoic acid esters, benzyl alcohol, para-chloro-meta-xylenol, chlorocresol, phenetyl alcohol, sorbic acid and salts thereof, thimerosal, chlorobutanol, and the like. The chelating agent is, for example, sodium edetate, sodium citrate or sodium salt of condensed phosphoric acid. In preparing the ophthalmic compositions in the form of eye ointments, the ointment base may be selected from among petrolatum, Macrogol, carboxymethylcellulose sodium, etc.

The ophthalmic compositions may be prepared by incorporating the active compound in a base or vehicle for topical application to the eye. To prepare a liquid preparation, the concentration of the active ingredient may range from about 0.001% to about 10% and is typically in the range of about 0.01% to about 5%. An ointment may be prepared by using the active compound in a concentration from about 0.001% to about 10%, typically about 0.01% to about 5%. The ophthalmic composition of this disclosure may be administered in accordance with the following schedules. In the form of eye-drops, one to several drops per dose are instilled with a frequency of once to 4 times a day according to the clinical condition. Of course, the dosage may be adjusted according to symptoms. The ophthalmic composition may be used topically for the treatment of the eye without causing local irritant effects and produces beneficial effects surpassing those obtainable with the conventional drugs of the same type.

Bromfenac or its pharmacologically acceptable salt or a hydrate thereof, may also be formulated in a stability enhanced aqueous liquid preparation, such as an alkyl aryl polyether alcohol type polymer such as tyloxapol, or a polyethylene glycol fatty acid ester such as polyethylene glycol monostearate. These bromfenac ophthalmic compositions may potentially treat a broader patient population, have greater stability properties, and may require a lower concentration or less doses of bromfenac then previously known bromfenac compositions. Topical application to the eye of a therapeutically effective amount of a topical ophthalmic composition comprises bromfenac at a concentration of about 0.05% w/v to about 0.24% w/v.

Bromfenac or its pharmacologically acceptable salt or hydrate thereof is a NSAID that is effective against inflammatory diseases of anterior or posterior segment of the eye, such as blepharitis, conjunctivitis, scleritis, and postoperative inflammation in the field of opthalmology, and its sodium salt has been practically used in the form of eye drops (“New Drugs in Japan, 2001”, 2001 Edition, Published by Yakuji Nippo Ltd., May 11, 2001, p. 27-29). The eye drop as mentioned above was designed to stabilize bromfenac by means of addition of a water-soluble polymer (e.g. polyvinylpyrrolidone, polyvinyl alcohol, etc.) and a sulfite (e.g., sodium sulfite, potassium sulfite, etc.) (Japanese patent No. 2,683,676 and its corresponding U.S. Pat. No. 4,910,225). In addition, an eye drop other than the above-mentioned one, Japanese patent No. 2,954,356 (corresponding to U.S. Pat. Nos. 5,603,929 and 5,653,972) discloses a stable ophthalmic composition which comprises incorporating an antibacterial quaternary ammonium polymer and boric acid into an acidic ophthalmic agent. The acidic agent described therein includes, for example, 2-amino-3-(4-bromobenzoyl)-phenylacetic acid.

Nevanac® (nepafenac ophthalmic suspension) 0.1% is a sterile, topical, nonsteroidal anti-inflammatory (NSAID) prodrug for ophthalmic use. Each mL of Nevanac® suspension contains 1 mg of nepafenac. Nepafenac is designated chemically as 2-amino-3-benzoyl-benzeneacetamide with an empirical formula of C₁₅H₁₄N₂O₂. The structural formula of nepafenac is:

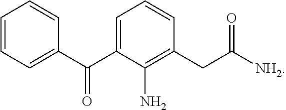

Nepafenac is a yellow crystalline powder. The molecular weight of nepafenac is 254.28. Nevanac® ophthalmic suspension is supplied as a sterile, aqueous 0.1% suspension with a pH approximately of 7.4. The osmolality of Nevanac® ophthalmic suspension is approximately 305 mOsmol/kg. Each mL of Nevanac® contains as the active ingredient: nepafenac 0.1%; and the inactive ingredients: benzalkonium chloride 0.005% (preservative), mannitol, carbomer 974P, sodium chloride, tyloxapol, edetate disodium, sodium hydroxide and/or hydrochloric acid to adjust pH and purified water, USP.

Ocufen® (flurbiprofen sodium ophthalmic solution, USP) 0.03% is a sterile topical nonsteroidal anti-inflammatory product for ophthalmic use. It's chemical name is sodium (±)-2-(2-fluoro-4-biphenylyl)-propionate dihydrate and has structural formula:

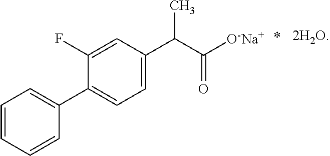

Ocufen® contains as the active ingredient: flurbiprofen sodium 0.03% (0.3 mg/mL); and the inactive ingredients: thimerosal 0.005% (preservative), citric acid; edetate disodium; polyvinyl alcohol 1.4%; potassium chloride; purified water; sodium chloride; and sodium citrate, and may also contain hydrochloric acid and/or sodium hydroxide to adjust the pH. The pH of Ocufen® ophthalmic solution is 6.0 to 7.0 and has an osmolality of 260-330 mOsm/kg.

### NSAIDs as Adjuvants to VEGF Inhibitors

Adjuvants are drugs that are given with other drugs at about the same time, which may have a different but complimentary therapeutic mechanism of action that may increase the efficacy or potency of the other drugs. Thus, the disclosure provides ophthalmic NSAIDs, including but not limited to Acular® (ketorolac tromethanine 0.5%, Allergan, Inc.); Xibrom® (bromfenac 0.09%, Ista Pharmaceuticals, Inc.); Nevanac® (nepanac 0.1% suspension, Alcon, Inc.); Ocufen® (flurbiprofen sodium 0.03%, Novartis AG), as adjuvants in combination with inhibitors of VEGF, such as Avastin® (bevacizumab), Lucentis® (ranibizumab injection), and Macugen® (pegaptanib sodium injection), in the treatment of wet AMD. The disclosure further provides methods for treating retinal disorders including but not limited to wet AMD, diabetic retinopathy, diabetic macular edema, central retinal vein occlusion, and branch retinal vein occlusion by administering one or more ophthalmic NSAIDs as an adjuvant to a patient in need thereof who is undergoing treatment with one or more VEGF inhibitors.

The adjuvant ophthalmic NSAIDs may be administered concurrently, pre- or post-treatment with one or more VEGF inhibitors. The NSAID may be applied topically to the eye in an ophthalmogically acceptable formulation in the form of an aqueous suspension or solution, an ointment, a gel or an aqueous solution which gels on contact with the eye. An aqueous solution or suspension is the typical formulation and has about 1 to about 15 mg of NSAID per ml of formulation. Alternatively, the NSAID may be administered concurrently with the intravitreal administration of one or more VEGF inhibitors directly into the eye.

The disclosed methods may include a dosing regime of once, twice, or up to six times daily administration into the eye. The dosing may be once a day with a higher dose NSAID formulation and twice a day with a lower dose NSAID formulation.

The disclosure also includes therapeutic methods wherein the retinal disorder is caused by surgery, physical damage to the eye, glaucoma, macular degeneration, or diabetic retinopathy. A still further aspect of the disclosure provides that the retinal disorder or injury is one caused by vascular leakage in the eye or by inflammation in the eye. Examples of conditions related to inflammation in the eye include, but are not limited to the following: surgical trauma, dry eye, allergic conjunctivitis, viral conjunctivitis, bacterial conjunctivitis, blepharitis, anterior uveitis, injury from a chemical, radiation or thermal burn, or penetration of a foreign body.

An additional aspect of the disclosure includes methods for treating retinal disorders, including but not limited to one or more additional active ingredients as part of the formulation. Such additional actives may include, but are not limited to, antihistamines and/or antibacterials and/or antimicrobial compounds, to further assist with the treatment of the retinal disorder.

An additional aspect of the disclosure provides methods for treating a retinal disorder wherein its normal condition has been disrupted or changed comprising administering to the eye one to six times daily the selected formulation.

The NSAID ophthalmic composition or formulation of the disclosure may be administered to a patient which is or may be suffering from an ophthalmic injury, surgery, disease or disorder (e.g., human, rat, mouse, rabbit, dog, cat, cattle, horse, monkey). The composition or formulation is given in an amount sufficient to cure, treat, or at least partially arrest the symptoms or complications of the ocular surgery, injury, disease or disorder. Amounts effective for this use will depend on the severity and course of the surgery, injury, disease or disorder, the patient's health status and response to the composition or formulation, and the judgment of the treating physician.

The formulation of the disclosure and its subsequent administration is within the skill of those in the art. Dosing is dependent on severity and responsiveness of the disease state to be treated, with the course of treatment lasting from one day to several months, or until a cure is effected or a diminution of the disease state is achieved. Optimal dosing schedules may be calculated from measurements of drug accumulation in the body of the patient.

An exemplary dosing schedule would comprise pretreating a patient from 48 hours to immediately before, during or after a scheduled ophthalmic procedure such as treatment for wet-AMD, and then optionally continuing treatment one or two times daily for approximately 14 days or until a physician is satisfied that the condition has been sufficiently corrected.

In cases where the formulation is used to treat a condition that is unscheduled, treatment may begin immediately from onset of any symptoms of whatever condition, disease or disorder is to be treated, and treated once or twice daily for approximately 14 days afterwards or until a physician is satisfied that the condition has been sufficiently corrected.

In addition to the medicament, flocculating and deflocculating agents and water, conventional excipients and other materials are advantageously employed in preparing the ophthalmic suspension compositions of the disclosure in accordance with good pharmaceutical practice. For example, the ophthalmic suspensions are sterile and typically contains a bacteriological preservative to maintain sterility during use. Quarternary ammonium bacteriostats such as benzalkonium chloride may be used as well as phenyl mercuric acetate, phenyl mercuric nitrate, thimerosal, benzyl alcohol, or β-phenylethyl alcohol. These bacteriostats may suitably be used in a range of from 0.01 to 3.0 mg/ml of total suspension. An anti-oxidant may also be used to prevent oxidation of the medicament. Suitable anti-oxidants include sodium bisulfite, N-acetyl cysteine salts, sodium ascorbate, sodium metabisulfite, sodium acetone bisulfite and other acceptable anti-oxidants known to the pharmaceutical art. These anti-oxidants may suitably be used in a range of 0.1 to 10.0 mg/ml. In conjunction with the anti-oxidants, chelating agents such as disodium edetate may also be employed.

Viscosity inducing agents helpful in suspension characteristics of the composition, including but not limited to cellulose derivatives such as hydroxymethyl cellulose, hydroxypropyl cellulose and methyl cellulose, may also be used in the formulations. For this purpose, one may use from 1.5 to 10.0 mg/ml of such agents. Lecithin may also be used to provide helpful suspension characteristics for the ophthalmic suspension composition, being employed for this purpose in amounts of from 0.05 to 1.0 mg/ml of total suspension. A humectant may also be used to help retain the water of the formulation in the eye. High molecular weight sugars are suitably used for this purpose such as sorbitol and dextrose in a concentration of from 0.1 to 10.0 mg/ml. Since the formulation may be autoclaved to obtain initial sterility an autoclaving aid such as sodium chloride may be added to the formulation.

The ophthalmic formulations of the disclosure include a NSAID or a derivative thereof as an active agent, a stabilizing agent (such as polyvinylpyrrolidone), a solubilizing agent (such as tyloxapol), a chelating agent (such as ethylenediaminetetraacetic acid (EDTA)), a preservative (such as benzalkonium chloride), a buffer (such as boric acid and sodium borate), a tonicity agent (such as sodium chloride) and optional additional active agents, viscosity/osmolality/pH enhancing agents, and various excipients.

A lower dose formulation of the disclosure comprises a NSAID or a derivative thereof at a concentration of about 0.05% w/v to about 0.1% w/v; polyvinylpyrrolidone at a concentration of about 0.35% w/v to about 3.00% w/v; a solubilizing agent at a concentration of about 0.002% w/v to about 0.2% w/v; a chelating agent at a concentration of about 0.005%, 10 w/v to about 0.1% w/v; a preservative at a concentration of about 0.0025% w/v to about 0.02% w/v; a tonicity agent at a concentration of about 0.08% w/v to about 0.14% w/v; wherein the final osmolality is about 250 to about 350 mOsm; and a buffering agent; wherein the final pH of the formulation is about 8.0 to about 8.5. A further aspect of the disclosure utilizes an alkyl aryl polyether alcohol type polymer as the solubilizer, EDTA as the chelating agent at a concentration of about 0.005% w/v to about 0.1% w/v, and/or benzalkonium chloride (BAK) as the preservative at a concentration of about 0.0025% w/v to about 0.02% w/v. A further aspect includes tyloxapol as the alkyl aryl polyether alcohol type polymer as the solubilizer in the formulation.

Another aspect of the disclosure provides a lower dose topical ophthalmic formulation comprising a NSAID or a derivative thereof at a concentration of about 0.05% w/v to about 0.1% w/v; boric acid at a concentration of about 0.8% w/v to about 1.4% w/v; sodium borate at a concentration of about 0.8% w/v to about 1.4% w/v; benzalkonium chloride at a concentration of about 0.0025% w/v to about 0.02% w/v; polyvinylpyrrolidone at a concentration of about 0.35% w/v to about 3.00% w/v; EDTA at a concentration of about 0.005% w/v to about 0.1% w/v; tyloxapol at a concentration of about 0.002% w/v to about 0.2% w/v; sodium chloride at a concentration of about 0.08% w/v to about 0.14% w/v; wherein the final pH of the formulation is about 8.0 to about 8.5. A further aspect of the formulation would have a final pH of about 8.3. A further aspect of the formulation comprises the final formulation in an aqueous formulation.

Another aspect of the disclosure provides a lower dose topical ophthalmic formulation comprising a NSAID or a derivative thereof at a concentration of about 0.08% w/v; boric acid at a concentration of about 1.1% w/v; sodium borate at a concentration of about 1.1% w/v; benzalkonium chloride at a concentration of about 0.005% w/v; polyvinylpyrrolidone at a concentration of about 2.00% w/v; EDTA at a concentration of about 0.02% w/v; tyloxapol at a concentration of about 0.02% w/v; sodium chloride at a concentration of about 0.109% w/v; and wherein the final pH of the formulation is about 8.3.

A higher dose formulation of the disclosure comprises a NSAID or a derivative thereof at a concentration of about 0.12% w/v to about 0.24% w/v; polyvinylpyrrolidone at a concentration of about 0.35% w/v to about 3.00% w/v; a solubilizing agent at a concentration of about 0.002% w/v to about 0.2% w/v; a chelating agent at a concentration of about 0.005% w/v to about 0.1% w/v; a preservative at a concentration of about 0.0025% w/v to about 0.02% w/v; a tonicity agent at a concentration of about 0.04% w/v to about 0.14% w/v; wherein the final osmolality is about 250 to about 350 mOsm; and a buffering agent; wherein the final pH of the formulation is about 7.6 to about 8.0. A further aspect of the disclosure utilizes an alkyl aryl polyether alcohol type polymer as the solubilizer, EDTA as the chelating agent at a concentration of about 0.005% w/v to about 0.1% w/v, and/or benzalkonium chloride (BAK) as the preservative at a concentration of about 0.0025% w/v to about 0.02% w/v. A further aspect includes tyloxapol as the alkyl aryl polyether alcohol type polymer as the solubilizer in the formulation.

Another aspect of the disclosure provides a higher dose topical ophthalmic formulation comprising a NSAID or a derivative thereof at a concentration of about 0.12% w/v to about 0.24% w/v; boric acid at a concentration of about 0.9% w/v to about 1.7% w/v; sodium borate at a concentration of about 0.4% w/v to about 1.0% w/v; benzalkonium chloride at a concentration of about 0.0025% w/v to about 0.02% w/v; polyvinylpyrrolidone at a concentration of about 0.35% w/v to about 3.00% w/v; EDTA at a concentration of about 0.005% w/v to about 0.1% w/v; tyloxapol at a concentration of about 0.002% w/v to about 0.5% w/v; sodium chloride at a concentration of about 0.04% w/v to about 0.14% w/v; wherein the final pH of the formulation is about 7.6 to about 8.0. A further aspect of the present formulation would have a final pH of about 7.8. A further aspect of the present formulation comprises the final formulation in an aqueous formulation.

Another aspect of the disclosure provides a higher dose topical ophthalmic formulation comprising a NSAID or a derivative thereof at a concentration of about 0.18% w/v; boric acid at a concentration of about 1.30% w/v; sodium borate at a concentration of about 0.74% w/v; benzalkonium chloride at a concentration of about 0.005% w/v; polyvinylpyrrolidone at a concentration of about 2.00% w/v; EDTA at a concentration of about 0.02% w/v; tyloxapol at a concentration of about 0.02% w/v; sodium chloride at a concentration of about 0.087% w/v; and wherein the final pH of the formulation is about 7.8.

Tyloxapol is an example of an isotonic surfactant which may function as a stabilizing, solubilizing or dispersing agent in the present formulation. The formulation may contain an alkyl aryl polyether alcohol type polymer such as tyloxapol which serves as a solubilizer in the formulation. Some of the properties related to alkyl aryl polyether alcohol type polymers, such as tyloxapol, in relation to stabilizing ophthalmic compositions is described in U.S. Patent Application Publication Number 2005/0239895, and is herein incorporated by reference. The formulation of the disclosure may include an alkyl aryl polyether alcohol type polymer at a concentration of about 0.002% w/v to about 0.5% w/v. Another aspect of the disclosure comprises tyloxapol at a concentration of about 0.002% w/v to about 0.2% w/v, and typically at a concentration of about 0.02% w/v.

Additional ophthalmic formulations of the disclosure include a NSAID or a derivative thereof as an active agent, a stabilizing agent (such as polyvinylpyrrolidone), a solubilizing agent (such as tyloxapol), a chelating agent (such as ethylenediaminetetraacetic acid (EDTA)), a preservative (such as benzalkonium chloride), a buffer (such as boric acid and sodium borate), sodium sulfite and optional additional active agents, viscosity/osmolality/pH enhancing agents, and various excipients.

Another aspect of the disclosure provides a lower dose formulation comprising a NSAID or a derivative thereof at a concentration of about 0.05% w/v to about 0.1% w/v; polyvinylpyrrolidone at a concentration of about 0.35% w/v to about 3.00% w/v; a solubilizing agent at a concentration of about 0.002% w/v to about 0.2% w/v; a chelating agent at a concentration of about 0.005% w/v to about 0.1% w/v; a preservative at a concentration of about 0.0025% w/v to about 0.02% w/v; sodium sulfite at a concentration of about 0.02% w/v to about 0.5% w/v; wherein the final osmolality is about 250 to about 350 mOsm; and a buffering agent; wherein the final pH of the formulation is about 8.0 to about 8.5. A further aspect of the disclosure utilizes an alkyl aryl polyether alcohol type polymer as the solubilizer, EDTA as the chelating agent at a concentration of about 0.005% w/v to about 0.1% w/v, and/or benzalkonium chloride (BAK) as the preservative at a concentration of about 0.0025% w/v to about 0.02% w/v. A further aspect includes tyloxapol as the alkyl aryl polyether alcohol type polymer as the solubilizer in the formulation.

Another aspect of the disclosure provides a lower dose topical ophthalmic formulation comprising a NSAID or a derivative thereof at a concentration of about 0.05% w/v to about 0.1% w/v; boric acid at a concentration of about 0.8% w/v to about 1.4% w/v; sodium borate at a concentration of about 0.8% w/v to about 1.4% w/v; benzalkonium chloride at a concentration of about 0.0025% w/v to about 0.02% w/v; polyvinylpyrrolidone at a concentration of about 0.35% w/v to about 3.00% w/v; EDTA at a concentration of about 0.005% w/v to about 0.1% w/v; tyloxapol at a concentration of about 0.002% w/v to about 0.2% w/v; sodium sulfite at a concentration of about 0.02% w/v to about 0.5% w/v; wherein the final pH of the formulation is about 8.0 to about 8.5. A further aspect of the present formulation would have a final pH of about 8.3. A further aspect of the present formulation comprises the final formulation in an aqueous formulation.

Another aspect of the disclosure provides a lower dose topical ophthalmic formulation comprising a NSAID or a derivative thereof at a concentration of about 0.08% w/v; boric acid at a concentration of about 1.1% w/v; sodium borate at a concentration of about 1.1% w/v; benzalkonium chloride at a concentration of about 0.005% w/v; polyvinylpyrrolidone at a concentration of about 2.00% w/v; EDTA at a concentration of about 0.02% w/v; tyloxapol at a concentration of about 0.02% w/v; sodium sulfite at a concentration of about 0.2% w/v; and wherein the final pH of the formulation is about 8.3.

Another higher dose formulation of the disclosure comprises a NSAID or a derivative thereof at a concentration of about 0.12% w/v to about 0.24% w/v; polyvinylpyrrolidone at a concentration of about 0.35% w/v to about 3.00% w/v; a solubilizing agent at a concentration of about 0.002% w/v to about 0.5% w/v; a chelating agent at a concentration of about 0.005% w/v to about 0.1% w/v; a preservative at a concentration of about 0.0025% w/v to about 0.02% w/v; sodium sulfite at a concentration of about 0.02% w/v to about 0.4% w/v; wherein the final osmolality is about 250 to about 350 mOsm; and a buffering agent; wherein the final pH of the formulation is about 7.6 to about 8.0. A further aspect of the disclosure utilizes an alkyl aryl polyether alcohol type polymer as the solubilizer, EDTA as the chelating agent at a concentration of about 0.005% w/v to about 0.1% w/v, and/or benzalkonium chloride (BAK) as the preservative at a concentration of about 0.0025% w/v to about 0.02% w/v. A further aspect includes tyloxapol as the alkyl aryl polyether alcohol type polymer as the solubilizer in the formulation.

Another aspect of the disclosure provides a higher dose topical ophthalmic formulation comprising a NSAID or a derivative thereof at a concentration of about 0.12% w/v to about 0.24% w/v; boric acid at a concentration of about 0.9% w/v to about 1.7% w/v; sodium borate at a concentration of about 0.4% w/v to about 1.0% w/v; benzalkonium chloride at a concentration of about 0.0025% w/v to about 0.02% w/v; polyvinylpyrrolidone at a concentration of about 0.35% w/v to about 3.00% w/v; EDTA at a concentration of about 0.005% w/v to about 0.1% w/v; tyloxapol at a concentration of about 0.002% w/v to about 0.5% w/v; sodium sulfite at a concentration of about 0.02% w/v to about 0.4% w/v; wherein the final pH of the formulation is about 7.6 to about 8.0. A further aspect of the present formulation would have a final pH of about 7.8. A further aspect of the present formulation comprises the final formulation in an aqueous formulation.

Another aspect of the disclosure provides a higher dose topical ophthalmic formulation comprising a NSAID or a derivative thereof at a concentration of about 0.18% w/v; boric acid at a concentration of about 1.30% w/v; sodium borate at a concentration of about 0.74% w/v; benzalkonium chloride at a concentration of about 0.005% w/v; polyvinylpyrrolidone at a concentration of about 2.00% w/v; EDTA at a concentration of about 0.02% w/v; tyloxapol at a concentration of about 0.3% w/v; sodium sulfite at a concentration of about 0.2% w/v; and wherein the final pH of the formulation is about 7.8.

The high dose formulation containing sodium sulfite may also contain an alkyl aryl polyether alcohol type polymer such as tyloxapol which serves as a solubilizer in the formulation. The high dose formulation containing sodium sulfite may include an alkyl aryl polyether alcohol type polymer at a concentration of about 0.002% w/v to about 0.5% w/v. Another aspect of the disclosure comprises tyloxapol at a concentration of about 0.002% w/v to about 0.5% w/v, and typically at a concentration of about 0.3% w/v.

The disclosed formulations may contain various excipients incorporated ordinarily, such as buffering agents (e.g., phosphate buffers, borate buffers, citrate buffers, tartarate buffers, acetate buffers, amino acids, boric acid, borax, sodium acetate, sodium citrate and the like), isotonicity agents (e.g., saccharides such as sorbitol, glucose and mannitol, polyhydric alcohols such as glycerin, concentrated glycerin, polyethylene glycol and propylene glycol, salts such as sodium chloride and potassium chloride, boric acid), preservatives or antiseptics (e.g., benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines and the like, chlorhexidine gluconate), solubilizing aids or stabilizing agents (e.g., cyclodextrins and their derivatives, water-soluble polymers such as polyvinylpyrrolidone, or carbomer, surfactants such as polysorbate 80 (Tween 80)), pH modifiers (e.g., hydrochloric acid, acetic acid, phosphoric acid, sodium hydroxide, potassium hydroxide, ammonium hydroxide and the like), thickening agents (e.g., polyvinylpyrrolidone, polyvinyl alcohol, sodium polyacrylate, hydroxyethyl cellulose, hydroxypropyl cellulose, methyl cellulose, hydroxypropylmethyl cellulose, carboxypropyl cellulose, carboxymethyl cellulose, and their salts), chelating agents (e.g., sodium edetate, sodium citrate, condensed sodium phosphate), antioxidant or radical scavenging agents (e.g., ascorbic acid (vitamin C) and its salts, tocopherol (vitamin E), and its derivatives, butylated hydroxy benzoic acids and their salts, 6-hydroxy-2,5,7,8-tetramethylchroman-2-carboxylic acid, gallic acid and its alkyl esters, uric acid and its salts and alkyl esters, sorbic acid and its salts, the ascorbyl esters of fatty acids, amines, sulfhydryl compounds (e.g., glutathione), and dihydroxy fumaric acid and its salts may be used, as well as EDTA (edetate, sodium edentate) BHT and the like. Descriptions of compounds used in standard ophthalmic formulations may be found in, for example, Remington's Pharmaceutical Sciences, latest edition, Mack Publishing Co., Easton, Pa.

Non-limiting examples of the contemplated excipients include a buffer, osmotic agent, demulcent, surfactant, emollient, tonicity agent, antioxidant and/or a preservative component.

The ophthalmic formulation when in an aqueous or non-aqueous form may also contain, but is not limited to: suspending agents (e.g., polyvinyl pyrrolidone, glycerin monostearate, sorbitan esters, lanolin alcohols) and dispersing agents (e.g., surfactants such as tyloxapol and polysorbate 80, ionic polymers such as sodium alginate) in addition to the agents listed above, to ensure that the ophthalmic formulation is satisfactorily dispersed in a uniform microparticulate suspension.

When the lower dose ophthalmic formulation is in the form of an aqueous suspension or solution, a non-aqueous suspension or solution, or a gel or ointment, typically a pH modifier may be used to give the formulation a pH between about 8.0 to 8.5, typically about 8.3. A pH modifier may be hydrochloric acid, sulfuric acid, boric acid, sodium borate, sodium hydroxide or any other ophthalmically-acceptable pH modifier.

When the higher dose ophthalmic formulation is in the form of an aqueous suspension or solution, a non-aqueous suspension or solution, or a gel or ointment, typically a pH modifier may be used to give the formulation a pH between about 7.6 to 8.0, more typically about 7.8. A pH modifier may be hydrochloric acid, sulfuric acid, boric acid, sodium borate, sodium hydroxide or any other ophthalmically-acceptable pH modifier.

Unless the intended purpose of use is affected adversely, the ophthalmic formulation of the disclosure may further comprise one or more additional therapeutically-active agents. Specific therapeutically-active agents include, but are not limited to: antibacterial antibiotics, synthesized antibacterials, antifungal antibiotics, synthesized antifungals, antineoplastic agents, steroidal anti-inflammatory agents, non-steroidal anti-inflammatory agents, anti-allergic agents, glaucoma-treating agents, antiviral agents, and anti-mycotic agents. Further contemplated are any derivatives of the therapeutically-active agents which may include, but not be limited to: analogs, salts, esters, amines, amides, alcohols and acids derived from an agent of the disclosure and may be used in place of an agent itself.

Examples of the antibacterial antibiotics include, but are not limited to: aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), β-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefrnetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Examples of the synthesized antibacterials include, but are not limited to: 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, 2-formylsulfisomidine, 4-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, 4-sulfanilamidosalicylic acid, 4-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibornol).

Examples of the antifungal antibiotics include, but are not limited to: polyenes (e.g., amphotericin b, candicidin, dennostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrroInitrin, siccanin, tubercidin, viridin).

Examples of the synthesized antifungals include, but are not limited to: allylamines (e.g., butenafine, naftifine, terbinafine), imidazoles (e.g., bifonazole, butoconazole, chlordantoin, chlormiidazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

Examples of the antineoplastic agents include, but are not limited to: antineoplastc antibiotics and analogs (e.g., aclacinomycins, actinomycin, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), antimetabolites exemplified by folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, gemcitabine, tagafur).

Examples of non-steroidal anti-inflammatory agents include, but are not limited to: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), E-acetamidocaproic acid, s-adenosylmethionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

Examples of anti-allergic agents include, but are not limited to: tranilast, ketotifen fumarate, pheniramine, diphenhydramine hydrochloride, sodium cromoglicate, bepotastine and bepotastine besilate.

Examples of glaucoma-treating agents include, but are not limited to: pilocarpine hydrochloride, latanoprost, timolol, iganipidine and isopropylunoprostone.

Examples of antiviral agents include, but are not limited to: idoxuridine, acyclovir, and trifluorouridine.

Examples of anti-mycotic agents include, but are not limited to: pimaricin, fluconazole, miconazole, amphotericin B, flucytosine, and itraconazole.

The active agent of the disclosure may be mixed with an ophthalmically acceptable carrier, excipient or diluent and formulated by a known method into a composition or formulation in various dosage forms such as injection solutions, eye drops, and ophthalmic gels or ointments. The formulation may be in a topical dosage form, typically an eye drop formulation in solution, or suspension form or an ophthalmic gel or ointment.

The ophthalmic formulations may, for example, be aqueous based such as aqueous eye drops, aqueous suspension eye drops, viscous eye drops and solubilized eye drops as well as non-aqueous based such as non-aqueous eye drops and non-aqueous suspension eye drops, or an ophthalmic gel or ointment.

An aqueous suspension typically contains sodium borate and boric acid as buffering agents, an alkyl aryl polyether alcohol type polymer such as tyloxapol as a stabilizing, solubilizing, dispersing or isotonicity agent, and polyvinyl pyrrolidone as a stabilizing agent.

An ophthalmic ointment may employ an ointment base known per se, such as purified lanolin, petrolatum, plastibase, liquid paraffin, polyethylene glycol and the like.

The disclosure also provides for an ophthalmic kit comprising the formulation disclosed herein and a means to apply the formulation to the eye. The ophthalmic kits may contain an application means which is an eye dropper, an eye cup, an eye spray or gel ointment tube and can comprise a single dose or a multi dose of the formulation in a single container.

### EXAMPLES

The following examples are offered to illustrate, but not to limit the claimed subject matter.

### Example 1

### A Combinatory Approach in the Treatment of Ocular Angiogenic Disease Using NSAIDs and Anti-Angiogenic Therapies

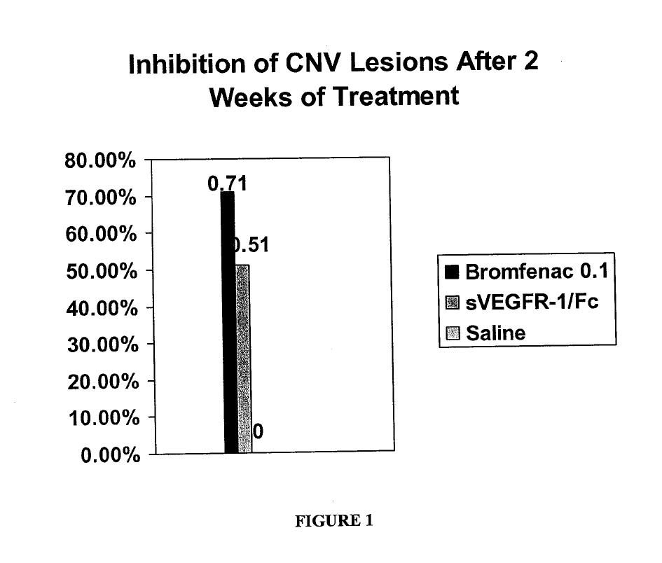

Recent data in a mouse model of retinal neovascular disease, shows a beneficial effect of bromfenac ophthalmic solution when given topically to mice with a laser induced angiomatous retina. As shown in FIG. 1, the anti-angiogenic effect produced by bromfenac was greater than that achieved with a soluble VEGF receptor administered intravitreally. FIG. 1 illustrates the inhibition of choroidal neovasularization (CNV) lesions after 2 weeks of treatment with topically applied bromfenac ophthalmic solution 0.1% (BF) on mice with CNV induced by laser photo coagulation; and the effect of BF 0.1% with vascular endothelial growth factor (VEGF)-neutralizing protein, recombinant murine soluble receptor 1/Fc chimeric protein (sVEGFR-1/Fc). The study shows that the area of choroidal neovascularization was reduced 71% by bromfenac treatment, compared to 51% with the soluble murine VEGF receptor. Since bromfenac is a highly selective COX-2 inhibitor, the anti-angiogenic effect seen with this chemical entity suggests that COX-2 is intimately involved in the retinal angiogenic disease process.

Jones et al., has shown that NSAIDs inhibit hypoxia-induced angiogenesis in vitro by upregulating von Hippel Lindau tumor suppressor. This in turn, increases the ubiquitination of HIF-α consequently targeting it for proteosome-mediated degradation. Loss of HIF-1α would lead to a loss of HIF-1α-activated gene transcription, one of which would be the VEGF-A gene. Thus, a COX inhibitor could give a reduction in all splice isoforms of VEGF-A.

The soluble VEGF receptor by definition, binds the secreted or extracellular-membrane bound forms of VEGF-A protein. The potential therefore exists, that these two agents (i.e., NSAIDS and VEGF inhibitors), one reducing mRNA pools of VEGF-A and the other depleting available pools of VEGF-A protein, might work in combination. Current therapies for ocular angiogenic diseases center on the administration of agents by intravitreal injection. The risks associated with this process are manifold and serious, including but not limited to endopthalmitis, retinal detachment and thromboembolic events in older at-risk patients. The above combinations would allow for a reduction in the number of intravitreal injections while preserving or enhancing the efficacy of the therapy. This may increase patient acceptance of this invasive therapy, reduce the impact on ophthalmic clinics with fewer injections per patient and may enhance patient-specific response compared to intravitreal VEGF treatment alone through the above combinatorial approach.

Purpose: To evaluate the inhibitory effect of topically applied bromfenac ophthalmic solution 0.1% (BF) on mice with choroidal neovascularization (CNV) induced by laser photo coagulation; and to compare the effect of BF 0.1% with vascular endothelial growth factor (VEGF)-neutralizing protein, recombinant murine soluble receptor 1/Fc chimeric protein (sVEGFR-1/Fc).

Methods: Six-week female C57BL/6J mice received laser burns with 514-argon laser at the posterior retina; 19 mice with successful burns were randomly assigned to 1 of 3 treatment groups: BF 0.1%, saline with or without 250 ng sVEGFR-1/Fc immediately after laser burn. Mice were treated with 4 μL of BF 0.1% or saline four times per day for 2 weeks. Then deeply anaesthetized mice were perfused with 50 mg/mL flourescein-labeled dextran, eyes were enucleated and fixed in buffered formalin. Flat mounts of the RPE-choroid-sclera were observed by fluorescence microscope. Total area of hyperfluorescent neovascualature was measured using image-analysis software

Results: Measurement of CNV area demonstrated the treatment for 2 weeks with Topical BF 0.1% ophthalmic solution or intravenous sVEGFR-1/Fc resulted in significantly smaller CNV lesions that that of saline. (71% and 51% respectively)

Conclusions: Topical bromfenac ophthalmic solution 0.1% significantly inhibited laser-induced CNV in mice. The degree of inhibition was greater than that of intravenously administered sVEGF-1/FC. This study suggests that the COX enzyme is strongly involved in promotion of CNV. BF may be a useful drug in the treatment of posterior ocular diseases associated with neovascularization.

All references cited herein are hereby incorporated by reference in their entireties, whether previously specifically incorporated or not. As used herein, the terms “a”, “an”, and “any” are each intended to include both the singular and plural forms.

Having now fully described the disclosed subject matter, it will be appreciated by those skilled in the art that the same may be performed within a wide range of equivalent parameters, concentrations, and conditions without departing from the spirit and scope of the disclosure and without undue experimentation. While this disclosure has been described in connection with specific embodiments thereof, it will be understood that it is capable of further modifications. This application is intended to cover any variations, uses, or adaptations of the subject matter following, in general, the principles of the disclosure and including such departures from the disclosure as come within known or customary practice within the art to which the subject matter pertains and as may be applied to the essential features hereinbefore set forth.

What is claimed is:

1. A method for increasing the interval between intravitreal injections in a patient undergoing treatment for a retinal disorder with a VEGF inhibitor to maximize visual acuity, the method comprising the step of administering to the patient in need of such treatment, an effective amount of an adjuvant comprising one or more ophthalmic NSAIDs.

2. The method of claim 1, wherein the retinal disorder is wet AMD, diabetic retinopathy, diabetic macular edema, central retinal vein occlusion, or branch retinal vein occlusion.

3. The method of claim 1, wherein the NSAID is bromfenac, diclofenac, flurbiprofen, ketorolac, nepafenac, amfenac, or indomethacin.

4. The method of claim 3, wherein the NSAID is bromfenac.

5. The method of claim 1, wherein the VEGF inhibitor is bevacizumab, ranibizumab, or pegaptanib.

6. The method of claim 1, wherein the NSAID is topically administered to the eye.

7. The method of claim 1, wherein the NSAID is administered before, during or after administration of the VEGF inhibitor.

8. The method of claim 1, wherein the interval between intravitreal injections is increased by one or more months.

9. A method for decreasing the number of intravitreal injections in a patient undergoing treatment for a retinal disorder with a VEGF inhibitor to maximize visual acuity, the method comprising the step of administering to the patient in need of such treatment, an effective amount of an adjuvant comprising one or more ophthalmic NSAIDs.

10. The method of claim 9, wherein the retinal disorder is wet AMD, diabetic retinopathy, diabetic macular edema, central retinal vein occlusion, or branch retinal vein occlusion.

11. The method of claim 9, wherein the NSAID is bromfenac, diclofenac, flurbiprofen, ketorolac, nepafenac, amfenac, or indomethacin.

12. The method of claim 11, wherein the NSAID is bromfenac.

13. The method of claim 9, wherein the VEGF inhibitor is bevacizumab, ranibizumab, or pegaptanib.

14. The method of claim 9, wherein the NSAID is topically administered to the eye.

15. The method of claim 1, wherein the NSAID is administered before, during or after administration of the VEGF inhibitor.

16. The method of claim 9, wherein the number of intravitreal injections is decreased by about half.

17. A method for decreasing the amount of a VEGF inhibitor administered by intravitreal injection in a patient undergoing treatment for a retinal disorder with a VEGF inhibitor to maximize visual acuity, the method comprising the step of administering to the patient in need of such treatment, an effective amount of an adjuvant comprising one or more ophthalmic NSAIDs.

18. The method of claim 17, wherein the retinal disorder is wet AMD, diabetic retinopathy, diabetic macular edema, central retinal vein occlusion, or branch retinal vein occlusion.

19. The method of claim 17, wherein the NSAID is bromfenac, diclofenac, flurbiprofen, ketorolac, nepafenac, amfenac, or indomethacin.

20. The method of claim 19, wherein the NSAID is bromfenac.

21. The method of claim 17, wherein the VEGF inhibitor is bevacizumab, ranibizumab, or pegaptanib.

22. The method of claim 17, wherein the NSAID is topically administered to the eye.

23. The method of claim 17, wherein the NSAID is administered before, during or after administration of the VEGF inhibitor.

24. The method of claim 17, wherein the amount of the VEGF inhibitor administered by intravitreal injection is decreased by about half.

25. A method for decreasing the risk to a patient undergoing intravitreal treatment for a retinal disorder with a VEGF inhibitor to maximize visual acuity, the method comprising the step of administering to the patient in need of such treatment, an effective amount of an adjuvant comprising one or more ophthalmic NSAIDs.

26. The method of claim 25, wherein the retinal disorder is wet AMD, diabetic retinopathy, diabetic macular edema, central retinal vein occlusion, or branch retinal vein occlusion.

27. The method of claim 25, wherein the NSAID is bromfenac, diclofenac, flurbiprofen, ketorolac, nepafenac, amfenac, or indomethacin.

28. The method of claim 27, wherein the NSAID is bromfenac.

29. The method of claim 25, wherein the VEGF inhibitor is bevacizumab, ranibizumab, or pegaptanib.

30. The method of claim 25, wherein the NSAID is topically administered to the eye.

31. The method of claim 25, wherein the NSAID is administered before, during or after administration of the VEGF inhibitor.

32. The method of claim 25, wherein the risk is infection, pain, light sensitivity, vision changes, increased eye pressure, retinal detachment, vitreous floaters endopthalmitis, or thromboembolic events.
